(19) United States
(12) Patent Application Publication
(10) Pub. No.: US 2026/0148828 A1
(43) Pub. Date: May 28, 2026

(54) DEVICE RECOMMENDATIONS USING MACHINE LEARNING

(71) Applicants: RESMED DIGITAL HEALTH INC., San Diego, CA (US); RESMED PTY LTD., Sydney (AU)

(72) Inventors: Genevieve Claire ATKINSON, Sydney (AU); Tom VAN DAAL, San Diego, CA (US)

(21) Appl. No.: 19/122,805

(22) PCT Filed: Oct. 20, 2023
(86) PCT No.: PCT/US2023/077431

§ 371 Date: Apr. 21, 2025

Related U.S. Application Data
(60) Provisional application No. 63/380,507, filed on Oct. 21, 2022.

(51) Int. Cl. G16H 20/30

(52) U.S. Cl. CPC G16H 20/30

(57) ABSTRACT

Techniques for improved machine learning-based prediction of uplift measures are provided. User data for a user is accessed, the user data comprising a set of user characteristics and an indication of a first device category, of a plurality of device categories, currently used by the first user. A predicted uplift measure is generated for a second device category, of the plurality of device categories, by processing the set of user characteristics using a trained machine learning model. The predicted uplift measure is provided to the first user via a graphical user interface (GUI).

### CROSS-REFERENCE TO RELATED APPLICATIONS

This Application is a 371 national phase application of PCT Application No. PCT/US 2023/077431, filed on Oct. 20, 2023, which claims the benefit of and priority to U.S. Provisional Ser. No. 63/380,507 , filed on Oct. 21, 2022, the entire contents of which are incorporated herein by reference.

### BACKGROUND

### Field

Embodiments of the present invention generally relate to machine learning. More specifically, embodiments relate to using machine learning to generate device recommendations.

### Description of the Related Art

In a wide variety of technologies and environments, users must often choose between multiple alternative options (which are often mutually exclusive) based on limited information. Generally, there are varying amounts of sophistication and assistance for such selections, depending on the particular space. For example, users that wish to switch to a new computer or smartphone may rely on their experiences, online articles, and the like. Similarly, patients desiring to switch to new medical devices or therapies may rely on the suggestions of their medical provider, who in turn relies on their expertise and experience.

Conventional approaches for item recommendations are inherently subjective and inaccurate, resulting in users retaining or continuing to select and use suboptimal alternatives.

### SUMMARY

According to one embodiment presented in this disclosure, a method is provided. The method includes: accessing user data for a user, the user data comprising a set of user characteristics and an indication of a first device category, of a plurality of device categories, currently used by the user; selecting a first clustering model, from a plurality of clustering models, based on the first device category; assigning the user to one or more clusters in the first clustering model based on the set of user characteristics; generating a predicted uplift measure for a second device category, of the plurality of device categories, based on data points in the one or more clusters; and in response to determining that the predicted uplift measure satisfies one or more defined criteria, facilitating provisioning of the second device category for the user.

According to one embodiment presented in this disclosure, a method is provided. The method includes: accessing user data for a user, the user data comprising a set of user characteristics and an indication of a first positive airway pressure (PAP) device category, of a plurality of PAP device categories, currently used by the user for a PAP therapy; generating a predicted uplift measure for a second PAP device category, of the plurality of PAP device categories, by processing the set of user characteristics using a trained machine learning model; and providing the predicted uplift measure to a medical provider via a graphical user interface (GUI), wherein the medical provider is associated with the PAP therapy of the user.

According to one embodiment presented in this disclosure, a method is provided. The method includes: accessing user data for a user, the user data comprising a set of user characteristics and an indication of a first positive airway pressure (PAP) device category, of a plurality of PAP device categories, currently used by the user for a PAP therapy; generating a predicted uplift measure for a second PAP device category, of the plurality of PAP device categories, by processing the set of user characteristics using a trained machine learning model; and providing the predicted uplift measure to the user via a graphical user interface (GUI).

Other embodiments provide processing systems configured to perform the aforementioned methods as well as those described herein; non-transitory, computer-readable media comprising instructions that, when executed by one or more processors of a processing system, cause the processing system to perform the aforementioned methods as well as those described herein; a computer program product embodied on a computer readable storage medium comprising code for performing the aforementioned methods as well as those further described herein; and a processing system comprising means for performing the aforementioned methods as well as those further described herein.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIG. 1 depicts an example workflow for generating device switches using machine learning.

FIG. 2 depicts an example timeline to facilitate data collection to enable improved training of machine learning models for switch recommendations.

FIG. 3 depicts an example machine learning model architecture for improved switch recommendations.

FIG. 4 is a flow diagram depicting a method for training machine learning model(s) to generate switch recommendations.

FIG. 5 is a flow diagram depicting a method for training a set of clustering models to generate predicted uplift measures.

FIG. 6 is a flow diagram depicting a method for using machine learning to drive device switches.

FIG. 7 is a flow diagram depicting a method for using a set of clustering models to generate predicted uplift scores.

FIG. 8 is a flow diagram depicting a method for facilitating the provisioning of devices based on predicted uplift measures.

FIG. 9 is a flow diagram depicting a method for generating predicted uplift measures for PAP devices and medical providers.

FIG. 10 is a flow diagram depicting a method for generating predicted uplift measures for PAP devices and users.

FIG. 11 depicts an example computing device configured to perform various embodiments of the present disclosure.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Embodiments of the present disclosure provide apparatuses, methods, processing systems, and computer-readable mediums for machine learning (ML)-based device recommendations for users.

In some embodiments of the present disclosure, positive airway pressure (PAP) medical devices are used as example devices that can be suggested or evaluated using the ML techniques described. For example, in some embodiments, the user is a patient who uses a particular PAP machine (or category of machine) for sleep therapy, and the ML system can be used to evaluate or predict whether one or more other machines or categories would be more beneficial for the patient. However, embodiments of the present disclosure are readily applicable to a wide variety of environments and devices. Additionally, as used herein, a “device” may include a computational or electronic device, as well as any other item or service that can be used or consumed by a user. For example, the techniques described herein can be used to predict the improvements or benefits that might accrue to a user if they switch to a different type or category of any given item or device, such as a different book, a different type of toothpaste, a different operating system for their computer, a different personal service, and the like.

In some embodiments, ML model(s) are trained based on a variety of user data to predict uplift measures. As used herein, an “uplift measure” can generally include any score, value, or other measure indicating the effect (e.g., the benefits) of selecting a given alternative and/or switching from a current selection to the given alternative. For example, in the case of continuous PAP (CPAP) devices, the predicted uplift measure may include metrics such as the predicted change or decrease in in the patient's apnea-hypopnea index (AHI), the predicted amount of leak from the new CPAP mask, the predicted amount of time that the patient will use the new device (e.g., the number of hours per day), the sleep quality of the patient, and the like.

In some embodiments, the uplift measure is a relative value indicating the predicted change relative to the current status quo. For example, the predicted uplift measure may indicate the predicted increase in sleep quality (e.g., as a percentage or multiplier from the current sleep quality of the user). In some embodiments, the uplift measure may additionally or alternatively include absolute values indicating the predicted values for one or more variables, such as a predicted sleep quality score.

The specific user data that is evaluated as input to the model may vary depending on the particular task and implementation. For example, to predict uplift measures for PAP users, the model may be trained based on data from a defined period of time prior to the switch (e.g., seven days), such as, without limitation, the user's average usage duration (e.g., average number of hours per day), average AHI, average mask leak volume, average pressure settings (e.g., the amount of pressure used), average number of days per week that the user used the device, the number of times (per day) that the user used the device, and the like. As additional examples, the system may use attributes such as the user's age, the user's gender, the user's weight, the duration that has elapsed since the user first starting using the current device (e.g., the number of days), the baseline AHI of the user (e.g., determined when they started therapy), the results of any sleep tests performed for the user, and the like.

Similarly, the specific device types or categories used as output from the system may vary depending on the particular task and implementation. In some embodiments, a defined set or list of device types or categories can be used to classify the first or original device and the second or suggested device based on the device's characteristics, design, unique identifier, or other attributes. For example, for PAP users, the original and suggested devices may be classified as a “full face” category (e.g., indicating that the PAP mask covers both the mouth and nose of the user), a “nasal” device category (e.g., indicating that the mask covers only the nose), a “pillow” device category (e.g., a nasal mask with additional cushioning), an “oral” device category (e.g., a mask that covers only the mouth of the user, leaving the nose exposed), a “hybrid” or “combination” device category (e.g., a hybrid oral/nasal mask), and the like. There may generally be any number and variety of device types or categories used depending on the particular implementation.

In some embodiments, the system trains the ML model(s) to receive not only the user data, but also the user's current device category (e.g., the type or category of PAP mask that the user currently uses). In at least one embodiment, the model is further trained to generate a predicted uplift measure for each alternative device type or category (e.g., a first score for a first category of mask, a second score for a second category, and so on). This allows users to readily and rapidly identify the best alternative(s) to their current selection.

In at least one embodiment, the ML model comprises a set or ensemble of clustering models, where each respective clustering model corresponds to a respective device category. For example, the system may select or identify which clustering model to use based on which device category the user currently uses. This can allow for more targeted and accurate model predictions, as compared to conventional solutions.

Generally, by using embodiments of the present disclosure, the system is able to provide improved user outcomes (e.g., reduced difficulties, increased benefits, and the like) as compared to conventional systems. Further, in some embodiments, the particular architectures, preprocessing, and training techniques disclosed herein can improve model performance and accuracy (as compared to conventional approaches), reduce computational expense and/or latency of the training and/or inference process, and generally improve the operations of the machine learning system during training, inferencing, or both.

### Example Workflow for Generating Device Switches Using Machine Learning

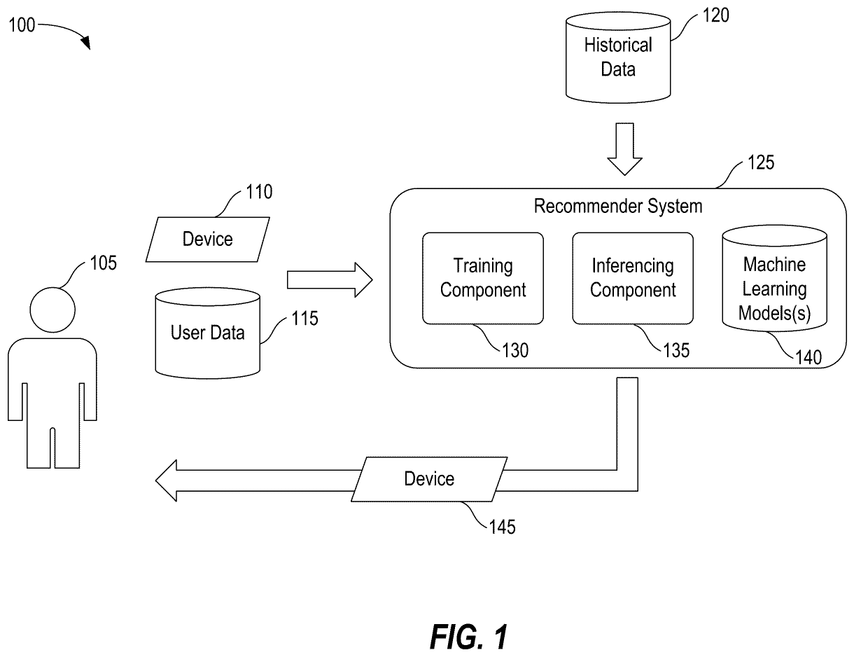

FIG. 1 depicts an example workflow 100 for generating device switches using machine learning.

In the illustrated example, information about a device 110 and user data 115 associated with a user 105 are provided to a recommender system 125, which uses one or more machine learning models 140 (trained based on historical data 120) to generate a suggestion or recommendation of a new device 145 for the user 105.

Though illustrated as a discrete system for conceptual clarity, in embodiments, the recommender system 125 may be implemented as a standalone system or device, or as part of a broader device or system. The operations of the recommender system 125 may be implemented using hardware, software, or a combination of hardware and software. In the illustrated example, the recommender system 125 includes a training component 130, an inferencing component 135, and a set of one or more machine learning models 140. Though depicted as discrete components for conceptual clarity, in embodiments, the operations of the training component 130 and inferencing component 135 may be combined or distributed across any number of components. Additionally, though depicted as residing in the recommender system 125 for conceptual clarity, in some embodiments, the machine learning models 140 may reside or be maintained in any suitable location and on any suitable system or device.

In the illustrated example, the recommender system 125 can train and/or refine the machine learning models 140 based on historical data 120 (e.g., using the training component 130), as well as use them for runtime inferencing to generate new recommendations based on input user data 115 (e.g., using the inferencing component 135). However, in some embodiments, the training and inferencing may be performed on separate systems. That is, one or more training systems or devices may be used to train the machine learning models 140, and the models may then be deployed to one or more inferencing systems or devices.

In the illustrated example, the training component 130 may access the historical data 120 to train and/or refine one or more machine learning models 140. As used herein, “accessing” data may generally include receiving, retrieving, requesting, or otherwise gaining access to the data. The historical data 120 generally includes information relating to prior device selections or switches. In one embodiment, the historical data 120 may include data points or exemplars, where each exemplar corresponds to a time when a user selected a device and/or changed or switched devices. For example, each exemplar may include or indicate a corresponding set of user attributes before, during, or after the switch, the first or original device that the user was using at the time of the switch, the second or new device to which the user switched, and the like.

For example, for CPAP devices, the historical data 120 may indicate the mask type or category that the user was currently using (e.g., a full-face mask type), attributes such as their AHI, mask leak, and the like for some period of time before the switch, an indication of the mask type or category to which the user switched (e.g., a nasal-only mask type), attributes such as their AHI, mask leak, and the like for some period after the switch, as well as general attributes such as their age, gender, weight, and the like. Although depicted as residing in a single repository or data source for conceptual clarity, in embodiments, the historical data 120 may generally include data stored, maintained, or provided from any number and variety of repositories and data sources.

Using the historical data 120, the training component 130 can iteratively train or refine the machine learning models 140. In at least one embodiment, the training component 130 can evaluate each exemplar to generate an uplift measure (also referred to as a historical uplift measure) for the device switch. Although referred to as an “uplift” measure for conceptual clarity, in embodiments, it is possible that a given metric worsened. For example, the volume of air leaking around the mask may increase, rather than decrease, after the switch. In some embodiments, these worse results are indicated using negative values for the uplift measure. For example, a value of 0.5 may indicate that the metric was 50% improved after the switch, while a value of −0.5 may indicate that the metric was 50% worse.

In an embodiment, the generated uplift measure can then be used as a target for training the machine learning model(s) 140, when given other user data (e.g., user attributes prior to the switch) as input. That is, the uplift measure may be generated based on one or more user attributes after the switch (either in isolation, or relative to the attributes prior to the switch), and this uplift can be used as the target model output when one or more user attributes from prior to the switch are provided as input.

Generally, the particular operations used to train the machine learning models 140 may vary depending on the particular implementation and model architecture. For example, in one embodiment, the model can include a neural network, and the training component 130 may train the network by providing attributes such as the user's gender, age, weight, and the like as input to the machine learning model 140 to generate one or more predicted uplift measures. These predictions can then be compared against the generated/ground-truth target measure in order to refine the model's accuracy (e.g., via backpropagation). As another example, for clustering models, the training component 130 may use one or more unsupervised ML techniques to cluster the exemplars in the historical data 120 based on the similarity of the attributes, and generate or assign the generated uplift measures to each cluster/exemplar appropriately, as discussed in more detail below.

Generally, the specific model architecture of the machine learning models 140 may vary depending on the particular implementation. For example, the machine learning models 140 may include regression models, classifier models, decision tree-based models, clustering models, and the like. In at least one embodiment, the training component 130 trains a set of models, where each model corresponds to a respective device category (from a defined list of alternative categories). For example, a first model corresponding to the “full-face mask” type may be trained using a subset of the historical data 120 (e.g., exemplars where the user's first or original device category was a “full-face mask”), such that the category-specific model can subsequently be selectively used when processing data from users during runtime, as discussed in more detail below.

Generally, the machine learning model(s) 140 may be trained to output a single predicted uplift measure (e.g., where a discrete model is trained specifically for each possible second or new device category, or where the models are trained to receive, as input, the proposed second or new device category) or a set of uplift measures (e.g., a predicted measure for each alternative category).

In some embodiments, the machine learning models 140 may be retrained according to a variety of training criteria, including periodically (e.g., weekly), upon determining that the model performance or accuracy has degraded (e.g., determined using test data or feedback from current users), and the like.

In the illustrated workflow 100, once the machine learning model(s) 140 are trained, the inferencing component 135 can use them to generate predicted uplift measures for new user data. As illustrated, user data 115 and an indication of a device 110 (e.g., a type or category of device) associated with a user 105 are accessed, by the inferencing component 135, for processing. Generally, the inferencing component 135 may access the user data 115 directly (e.g., where the user inputs or provides their information) or indirectly (e.g., where a healthcare provider or other third party provides or inputs the user's information).

In some embodiments, the particular contents of the user data 115 may vary depending on the particular implementation. In at least one embodiment, the information included in the user data 115 may have features or information corresponding to some or all of the data, from the historical data 120, used to train the models. For example, if the machine learning models 140 are trained to generate predictions based on attributes such as the user's age, AHI, mask leak, mask pressure, and the like, the inferencing component 135 may similarly identify, access, or extract values for these features or variables.

In some embodiments, the inferencing component 135 accesses the user data 115 (or the user data 115 is provided) because the user 105 (or a third-party representing or working on behalf of the user) has decided to investigate whether any alternative devices would benefit the user 105. For example, the user 105 may be dissatisfied with their CPAP machine, or the user's doctor or other healthcare service provider may determine that an alternative CPAP machine may be more beneficial. In response to this determination, the user 105 (or the service provider) may provide the user data 115 to the inferencing component 135, or otherwise initiate the inferencing process.

In some embodiments, the inferencing component 135 can additionally or alternatively access the user data 115 based on other triggering criteria. For example, the inferencing component 135 may periodically access and evaluate user data 115 for one or more users (e.g., weekly, monthly, yearly, and the like) to determine whether any device alternatives may be beneficial.

As discussed above and in more detail below, the inferencing component 135 can process the user data 115 and/or the type or category of the device 110 using one or more machine learning models 140 to generate one or more predicted uplift measures for one or more alternative device categories. Generally, the particular operations used to generate the predicted uplift measure(s) may vary depending on the particular model architecture and implementation.

For example, in some embodiments, the inferencing component 135 selects which model(s) to use based on the device 110, and processes the user data 115 using these selected model(s) to generate the predicted uplift measure(s). In other embodiments, the inferencing component 135 may provide the type or category of the device 110, alongside the user data 115, as input to the model(s) in order to generate predicted uplift measure(s). In some embodiments, each model may similarly output a single predicted uplift measure for a single alternative device category (e.g., where each model is trained for that specific category, or where the model is trained to receive the proposed alternative device as input alongside the user data 115), or a model may output multiple measures (e.g., one measure for each alternative device category).

In at least one embodiment, where the machine learning models 140 are cluster-based, the inferencing component 135 may select a clustering model based on the device 110 (e.g., selecting a model where the clusters were trained or generated based on historical data 120 indicating the same first device category). The inferencing component 135 can then assign the user 105 to one or more clusters based on their user data 115, where each cluster indicates the predicted uplift measure for one or more alternative device categories.

Irrespective of the particular implementation of the machine learning models 140, in the illustrated example, an indication of a new device 145 is returned to the user 105 (or to a third party, such as their healthcare provider). In some embodiments, the recommender system 125 outputs the device 145 based on determining that it was the highest-scored alternative (e.g., the device category having the highest predicted uplift measure). In at least one embodiment, the recommender system 125 can output multiple devices 145, each with a corresponding uplift measure. For example, the recommender system 125 may filter and/or rank the alternatives based on their predicted uplift, and output all or a subset of the alternatives. This may allow the user 105 (or third party) to quickly decide whether to switch to the new device 145. Generally, outputting the device 145 can include displaying it on a graphical user interface (GUI) of the recommender system 125, and/or transmitting or otherwise providing or indicating it to another system (e.g., a device used by the user 105 or a third-party, such as the user's doctor) for display via a GUI.

In at least one embodiment, the recommender system 125 may additionally or alternatively take other action, such as automatically provisioning or providing a device of the selected or suggested device category for the user 105. This provisioning may be performed upon receiving acceptance or approval of the proposal (e.g., from the user 105 for a third party), or automatically upon other criteria being satisfied (e.g., in response to determining that the predicted uplift is sufficiently high, or that the user 105 pre-approved the provisioning of whatever alternative was deemed most optimal).

For example, the recommender system 125 may perform operations such as creating a work order, submitting a purchase order, initiating shipping or other transportation, or otherwise performing any actions needed to actually provide the new device to the user 105.

### Example Workflow for Data Collection to Enable Improved Training of Machine Learning Models for Switch Recommendations

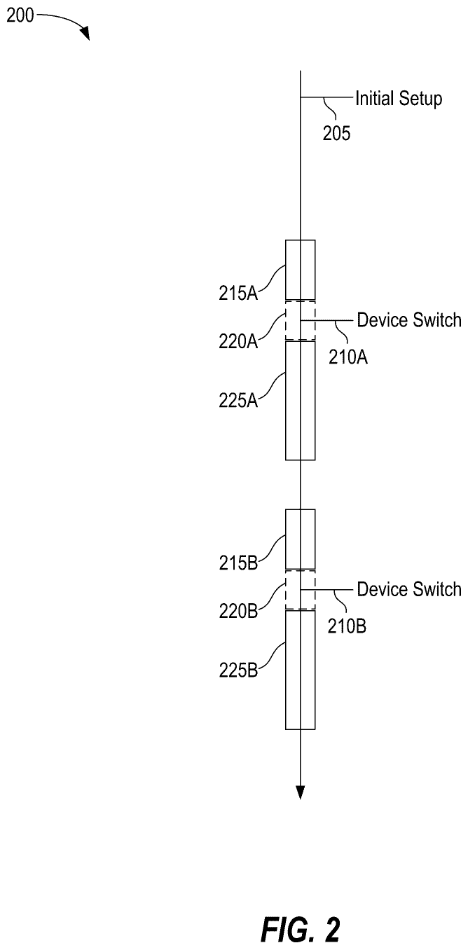

FIG. 2 depicts an example timeline 200 to facilitate data collection to enable improved training of machine learning models for switch recommendations.

In the illustrated example, the timeline 200 depicts a temporal ordering of events and data for a given user. For example, the timeline 200 may depict the events and relevant user data for a user that began, switched, and/or used a device (such as a PAP device) at some point in the past. In at least one embodiment, the timeline 200 corresponds to or depicts one or more exemplars from the historical data 120 of FIG. 1.

As illustrated by event 205 (labeled initial setup), at a first point in time, a user began using a device. Continuing the above-discussed PAP therapy example, the event 205 may correspond to the time when the user underwent the initial setup for the therapy, such as when they received or started using the prescribed PAP device. Generally, beginning at event 205 and continuing down the timeline 200, the user may be said to be “using” or otherwise “associated with” this initial device that was provided to them.

At event 210A (labeled device switch), at a subsequent point in time, the user switched from the initially-provided device to a new device. Generally, this switch may have been performed for any reason, such as preference, comfort, random whim, and the like. In an embodiment, the event 210A may be triggered by any event or criteria, including user action (e.g., where the user requests another device), by action of a third party (e.g., where the user's doctor or other healthcare provider suggests or provides a new device), based on other criteria (e.g., an expiration date of the original device), and the like. Generally, until at or near the event 210A, the user was using the initial device (provided at the initial setup event 205). Similarly, beginning at or near the event 210A, the user began using the newly-provided device.

In an embodiment, the particular techniques used to identify the event 210A may vary, depending on the particular implementation. For example, in some aspects, the recommender system may evaluate the historical data to find records or other data indicating when a new device was delivered to a user, or when a user picked up or otherwise acquired a new device.

In some embodiments, in response to identifying the event 210A in the historical data, a recommender system (e.g., recommender system 125 of FIG. 1) or another system can identify, extract, or otherwise access data surrounding this event 210A in order to generate a training exemplar, which can subsequently be used to train one or more machine learning models (e.g., machine learning models 140 of FIG. 1). In some embodiments, this extraction is performed for all identified device switch events 210. In at least one embodiment, the recommender system can first confirm that the switch event 210 satisfies some defined criteria prior to using the surrounding data for training.

For example, in one embodiment, the recommender system determines whether the event 210A is at least a defined duration from the initial setup event 205 (e.g., at least ten days from event 205). If not, the recommender system may determine to ignore or discard the surrounding data to improve model accuracy (e.g., to avoid using data which may be inaccurate or unreliable).

As another example, the recommender system may determine whether the event 210A corresponds to the acquisition of a single new device, as opposed to multiple. For example, if the user acquired a single new PAP mask, the recommender system may determine that the event 210A represents usable training data, because it can reasonably be inferred that the user switched to the single new mask. However, if the user ordered multiple PAP masks (e.g., to sample different categories to determine which they prefer), the recommender system may ignore or discard the surrounding data, as the new device cannot be confidently or reliably determined.

In an embodiment, as discussed above, if the event 210 satisfies the relevant criteria (if any), the recommender system extracts some set of user data from some period or window of time prior to the switch (e.g., prior to the identified device switch event 210A), as illustrated by block 215A, as well as a set of user data from a period or window of time subsequent to the switch (e.g., subsequent to the device switch event 210A), as illustrated by block 225A.

For example, as discussed above, the recommender system may extract, from the time or days corresponding to blocks 215A and 225A, the user's average usage duration, average AHI, average mask leak volume, average pressure settings, average number of days per week that the user used the device, average number of times (per day) that the user used the device, and the like. In an embodiment, the duration covered by the blocks 215A and 225A may be the same (e.g., both covering a seven day period) or may differ (e.g., where the block 215A covers a seven day period and the block 225A covers a fourteen day period), depending on the particular implementation.

In the illustrated example, there is also a blackout period (indicated by block 220A), where data is not collected or used by the recommender system to train the model(s). In some embodiments, the specific duration of the blackout period may vary depending on the particular implementation. Generally, a user or administrator may define the blackout period due to inherent uncertainty as to when the device switch actually occurs. For example, if the device switch is accomplished by shipping the new device to the user, the recommender system may know the date when the new device arrived, but be unable to determine the precise date when the user stopped using the old device and started using the new device.

In some embodiments, the blackout period (indicated by block 220A) is defined as a duration of time (e.g., three days) before and after the event 210A (which may be defined as the point or day in time when the new device was delivered, picked up, or otherwise accessed by the user). In some embodiments, the blackout period is defined as a duration of time subsequent to the event 210A (e.g., where the prior time represented by block 215A continues up until the event 210A, or until the day before the event 210A).

Advantageously, by using a blackout period, the recommender system can ensure that the data it extracts from the prior window (e.g., block 215A) and subsequent window (e.g., block 225A) is accurate (e.g., that it most likely represents use of the prior device and new device, respectively). This allows the model(s) to be trained with more reliable data, resulting in improved model performance and accuracy, as well as reduced computational expense (e.g., from processing or training using data with unclear reliability or accuracy).

Although the illustrated example depicts use of a blackout period in block 220A, in some embodiments, the recommender system can collect usage/user data from prior to and after the switch without such blackout period. For example, in one embodiment, using one or more sensor technologies or systems, the recommender system may be able to detect or identify the specific point or window in time where the user switched devices (rather than inferring the switch based on other data, such as device delivery). In one such embodiment, the recommender system can use this point as the event 210, and collect user data from neighboring or adjacent windows of time that share this event 210 as the border/delineation point.

That is, a given switch event 210 may be a determined/specific point in time when the user switched devices (e.g., determined using one or more sensors), such as a specific date, or may be an inferred or estimated point in time (e.g., estimated based on delivery confirmations), such as an estimated switch date.

In the illustrated timeline 200, the recommender system also detects another switch event 210B. As discussed above, in some embodiments, the recommender system can similarly determine whether this event satisfies one or more defined criteria (e.g., whether a single device was requested, whether the event 210B is sufficiently far from the earlier event 210A, and so on) before determining whether to use the surrounding data to train the model(s).

In the illustrated example, the recommender system can similarly extract the prior data (represented by block 215B) and subsequent data (represented by block 225B), ignoring or refraining from extracting and/or processing data from a defined blackout period (represented by block 220B) surrounding or associated with the event 210B. In this way, the recommender system may identify any number of switch events for a single user, generating a corresponding training exemplar for each.

As discussed above and in more detail below, the recommender system may compare the user data subsequent to each switch event 210 (e.g., block 225) with the user data prior to the switch event 210 (e.g., block 215) to generate the historical uplift measure for each switch event 210. Specifically, the data represented by block 215A may be compared against the data represented by block 225A in order to determine the uplift that was achieved by switching from the first device (provided at initial setup event 205) and the new device (provided at switch event 210A). Similarly, the data represented by block 215B may be compared against the data represented by block 225B in order to determine the uplift that was achieved by switching from the first new device (provided at switch event 210A) and the second new device (provided at switch event 210B).

As used herein, a device may generally be referred to as an “original,” “initial,” or “first” device to indicate that it was used prior to a given switch event. Similarly, a device may be referred to as a “new” or “second” device to indicate that it was used subsequent to the given switch event. In this way, a single device may be both a first device and a second device. For example, the device acquired at switch event 210A is a “second” device with respect to the event 210A, but a “first” device with respect to the event 210B.

### Example Machine Learning Model Architecture for Improved Switch Recommendations

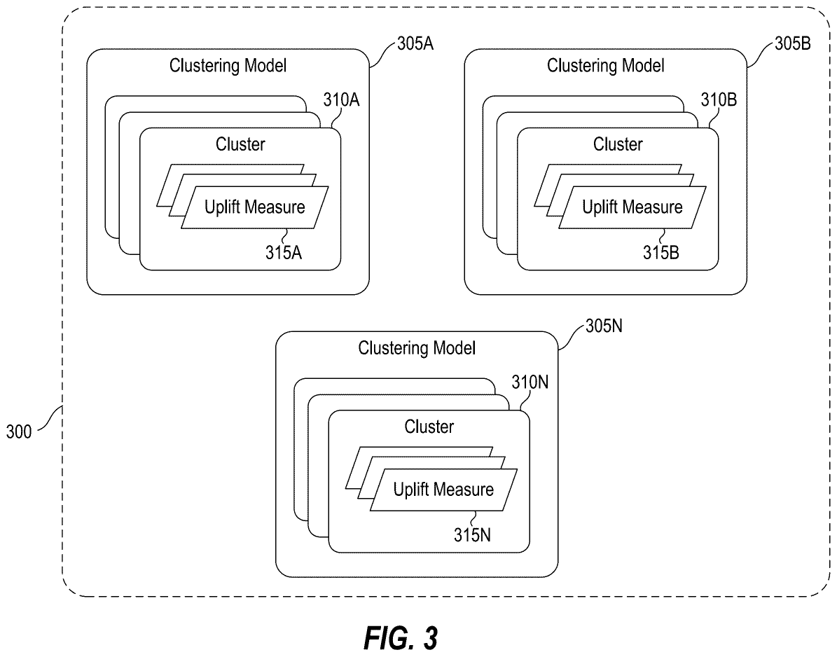

FIG. 3 depicts an example machine learning model architecture 300 for improved switch recommendations. In some embodiments, the architecture 300 corresponds to a machine learning model trained to generate predicted uplift measures based on device switches, such as the machine learning model 140 of FIG. 1. In some embodiments, the architecture 300 is used by a recommender system, such as recommender system 125 of FIG. 1, to facilitate provisioning of new devices to users.

As discussed above, in embodiments, the recommender system may use a wide variety of model architectures, such as regression models, depending on the particular implementation. The depicted architecture 300 depicts a clustering-based model. In the depicted architecture 300, the recommender system can train and/or use a machine learning model that itself comprises a set of clustering models 305A-N. That is, the model represented by architecture 300 may be an ensemble of individual clustering models 305.

In at least one embodiment, each clustering model 305 may correspond to a given first device category. That is, for each identified device switch (e.g., each event 210 of FIG. 2), the recommender system may categorize the corresponding data based on the first device that the user is ceasing to use. For example, if the user is switching away from a full-face PAP mask to some other mask category, the recommender system may use the corresponding data to train the clustering model 305A, regardless of what device the user is switching to. Similarly, if the user is switching away from a nasal mask category, the recommender system may use the corresponding data to train the clustering model 305B, regardless of which device category the user begins to use after the switch. In this way, at inference time, the recommender system can identify the appropriate clustering model 305, as discussed in more detail below.

In the illustrated example, each clustering model 305 includes a plurality of clusters 310. In an embodiment, the recommender system generates the clusters 310 by clustering users based on the extracted user data from prior to the switch. For example, for the clustering model 305A, the recommender system may identify a set of exemplars/switches where the first device was the device corresponding or assigned to the clustering model 305A. The recommender system may then use the prior data (e.g., represented by block 215 of FIG. 2) for each exemplar to cluster the users/device switches based on their initial (pre-switch) data (e.g., such that users with similar initial AHI, initial duration data, and the like are clustered together) into clusters 310A.

Generally, the recommender system can use a variety of clustering techniques or algorithms to generate the clusters 310. For example, in one embodiment, the recommender system uses a k-means clustering approach. In such an embodiment, each user or device switch may be assigned to a single cluster 310 (e.g., the one having the most-similar patients). In some embodiments, the recommender system uses a Gaussian mixture clustering approach to assign users/switch events to all clusters based on model probabilities. That is, if a Gaussian mixture approach is used, the recommender system may (at inference time), generate, for each respective cluster, a probability that the user belongs to the respective cluster.

In some embodiments, to enable improved clustering, various preprocessing may be performed on the data. For example, in one embodiment, the recommender system may optionally apply one or more dimensionality reduction operations to the input data prior to clustering it. In one such embodiment, the recommender system reduces the dimensionality of the input data (e.g., the number of features) to two, which enables efficient visualization of the clusters 310. In some embodiments, applying the dimensionality reduction can additionally reduce the computational complexity of the clustering itself. For example, it may be more computationally efficient (e.g., require reduced resources) to first reduce the data dimensionality to a lower amount (e.g., to two dimensions) prior to clustering, as opposed to clustering on the relatively higher-dimensional data.

In some embodiments, the recommender system can optionally perform various operations to identify the optimal number of clusters 310 for each clustering model 305 (as many clustering techniques require that the user specify the number of clusters). In one such embodiment, the recommender system may use the same set of data (e.g., switches from a given first device category) to train multiple cluster models 305, each with a different number of clusters. The recommender system may then evaluate each such model (e.g., based on the silhouette score of each) to identify the optimal number of clusters for the specific set of data (e.g., for the given first device category). In this way, each clustering model 305 may use a different number of clusters, determined based on the underlying data used to train each.

In the illustrated example, each cluster 310 further includes a set of uplift measures 315. Generally, each respective uplift measure 315 indicates the historical or predicted uplift if the user switches to a respective device category (e.g., determined based on the data represented by blocks 225 of FIG. 2). For example, the recommender system may aggregate the historical uplift measures for a given set of data (e.g., a switch from a given first device, corresponding to the clustering model 305, to a given second device) to generate the predicted uplift for the specific switch. For example, for data relating to a switch from device A to device B, the recommender system may identify the clustering model 305 trained for initial device A. The recommender system can then use this data to train the identified clustering model 305. Then, for whichever specific cluster 310 the data was assigned to, the recommender system can update a corresponding uplift measure 315 to reflect that users that use initial device A and assigned to the specific cluster 310 (based on their characteristics) can expect an uplift similar to the historical uplift, if they switch to device B.

In an embodiment, at inferencing time, the recommender system can use the architecture 300 to efficiently generate predicted uplift measures for each user. For example, the recommender system may identify the first or current device category used by the user, and thereby identify the corresponding clustering model 305 trained based on data from other users that began with the device category. The recommender system may then assign the user to one or more clusters 310 from the clustering model 305 based on the user's current data (e.g., the average usage duration from the past seven days, the user's age, and the like). For example, in a k-means clustering approach, the user may be assigned to a single cluster 310. For a Gaussian mixture-based approach, the recommender system may assign the user to all clusters 310, with a determined probability for each.

Then, for each possible alternative or second device category, the recommender system can determine the corresponding uplift measure based on data points (e.g., exemplars) within the assigned cluster(s). In the case of a k-means system, the recommender system can simply determine and output the uplift measures 315 from the assigned cluster 310. In some embodiments, such as if a Gaussian mixture model is used, the recommender system can aggregate the uplift measures 315 from each cluster 310 based on the generated cluster probabilities. For example, the recommender system may generate, for each alternative second device category, a weighted average of the uplift measures 315, where the weight corresponds to the cluster probability generated for the user.

In this way, the architecture 300 enables the recommender system to efficiently train and use machine learning model(s) to generate predicted uplift measures for users. This can substantially improve results and model accuracy while minimizing computational resources and latency.

### Example Method for Training Machine Learning Model(s) to Generate Switch Recommendations

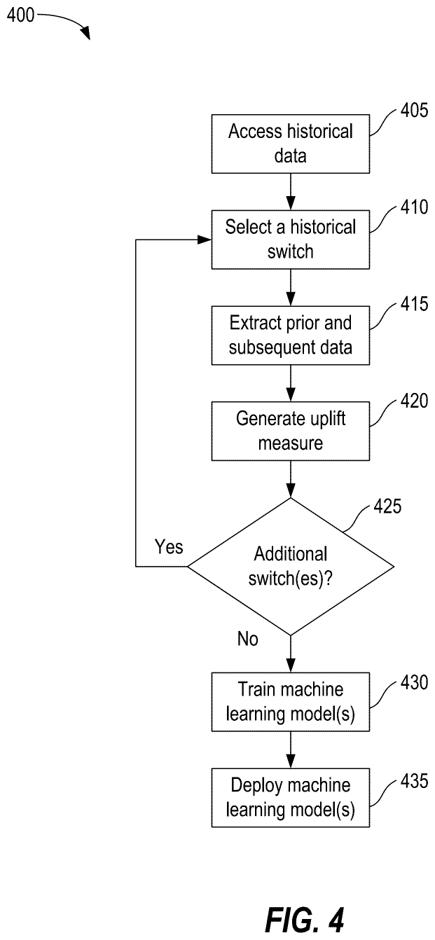

FIG. 4 is a flow diagram depicting a method 400 for training machine learning model(s) to generate switch recommendations. In some embodiments, the method 400 is performed by a recommender system, such as the recommender system 125 of FIG. 1.

At block 405, the recommender system accesses historical data (e.g., historical data 120 of FIG. 1). As discussed above, the historical data generally includes information relating to prior device (or other item) usage and switches for any number of users and across any amount of time. For example, in at least one embodiment, the historical data includes therapy data for a number of patients or users of PAP devices, such as information relating to when they began therapy, their initial attributes at that time (e.g., their age, their initial pre-therapy AHI, and the like). In an embodiment, the historical data further indicates which device category(s) or categories the user has used, as well as when the user used them.

For example, the historical data may include information relating to event(s) or points in time when a given user requested, received, or otherwise gained access to a new device, as well as an indication of the category of the new device. As discussed above, the device category can generally correspond to any categorization or characteristic of the device. For example, for PAP devices, the category may correspond to the way the face mask fits the user's face (e.g., full-face, nasal, pillow, oral, hybrid, and the like). As additional examples, the “category” of the object may correspond to concepts like the genre or style in general, or to more specific identifiers such as the specific model, a specific book, and the like.

In an embodiment, the historical data includes user data relating to the use of the device(s). Generally, the specific data included may vary depending on the particular implementation and task. For example, if the recommender system is training a model to suggest smartphone devices, the usage information can include data such as how long the user has possessed the device and their average usage (e.g., hours per day, average battery life, computational resources used, network bandwidth used, and the like), as well as other information such as the user's age. As another example, for PAP systems, the historical data may include information such as the user's AHI at one or more points in time, usage duration(s) for one or more days or windows of time, pressure settings, and the like.

In some embodiments, the historical data includes all the user/usage data that is available for each user (e.g., since they began use). In at least one embodiment, the historical data may selectively include such data only in relation to device switches (e.g., only within a defined window of time before and/or after a switch event). For example, the recommender system may continually collect such usage data, and discard it after a defined period of time if the user has not switched to a new device (e.g., discarding any data older than seven days, or the duration defined for collecting data prior to a switch). This can enhance user privacy and reduce the computational expense of storing or maintaining the data.

At block 410, the recommender system identifies and/or selects one of the historical switches reflected in the historical data. For example, the recommender system may parse the historical data for one or more users to identify events (e.g., device switch events 210 of FIG. 2) when the user switched to or acquired a new device or device category. As discussed above, the particular techniques used to identify the historical switches may vary depending on the particular implementation, and may include identifying events such as the shipping of the object, the delivery of the object, the pickup of the object from a physical location, the download and/or installation of new software or data, and the like.

Generally, any suitable technique can be used to select the historical switch at block 410, as the recommender system will evaluate each historical switch in the historical data during the method 400. For example, the recommender system may select the switches randomly or pseudo-randomly, may select all the switches for a single user before moving to the next, may select the switches in sequential order regardless of the associated user, and the like. Although the illustrated example depicts a sequential process (e.g., selecting and evaluating each historical switch in turn) for conceptual clarity, in some embodiments, the recommender system may select and process some or all of the historical switches in parallel.

At block 415, the recommender system extracts, from the historical data, prior user data and subsequent user data with respect to the selected switch. For example, as discussed above, the recommender system may extract the relevant user data or features (e.g., corresponding to the features used to train the model(s)) from a defined window prior to the switch (e.g., a seven day period, which may end on the date of the switch or a defined number of days prior to the switch), as well as the relevant user data or features from a defined window subsequent to the switch (e.g., a fourteen day period, which may begin on the date of the switch event or a defined number of days after the switch).

For example, as discussed above, for a PAP therapy solution, the recommender system may extract and/or generate information based on data collected during the window, such as the average usage duration per day, the current or average AHI of the user, a total or average amount of mask leak during the window, pressure data (e.g., the average pressure the PAP machine used), and the like. In some embodiments, as discussed above, the recommender system may further extract user data unrelated to the specific device switch, such as the user's age, gender, weight, and the like.

Although not included in the illustrated example, in at least one embodiment, the recommender system can optionally evaluate the selected switch using one or more criteria prior to extracting information for it. For example, the recommender system may confirm that the user did not acquire more than one new device category at the same time (or within a defined window of time from the selected switch), that the switch is sufficiently far (in time) from one or more other defined events, and the prior and/or subsequent data meets one or more criteria (such as a minimum amount or quality of the data), and the like.

At block 420, the recommender system determines or generates an uplift measure for the selected historical switch based on the extracted data. As discussed above, the recommender system may generate the uplift measure by comparing the data that was subsequent to the switch with the data that was prior to the switch, and identifying features that improved, worsened, or remained the same (as well as the amount of each change). The uplift measure can include absolute values (e.g., the absolute magnitude of the change and/or the direction of the change), and/or relative values (e.g., the amount of change, relative to the prior values). For example, the uplift measure may indicate that the usage duration increased by two hours and/or by 20%.

Generally, as discussed above, the particular information the uplift measure represents may vary depending on the particular implementation and task. For example, for PAP therapies, the uplift measure may indicate the change in AHI, the change in mask leak, the change in usage duration, the change in sleep quality, the change in user-reported comfort or satisfaction with the device or therapy, and the like. As discussed above and in more detail below, this uplift measure can be used as the target/output variable (e.g., the ground-truth label) when training the machine learning model(s). In this way, the recommender system can generate a training exemplar that includes at least the user data from prior to the switch, with the generated uplift measure(s) as the target/label.

At block 425, the recommender system determines whether there is at least one additional historical switch, in the historical data, that has not-yet been evaluated to generate a training exemplar. If so, the method 400 returns to block 410. If not, the method 400 continues to block 430.

At block 430, the recommender system trains one or more machine learning model(s) based on the training exemplars (e.g., based on the prior user data for each switch and the corresponding uplift measure generated at block 420). Generally, the particular training process may vary depending on the particular implementation and model architecture. For example, to train an artificial neural network model, the recommender system may provide the user data (from prior to a given switch) as input to the model to generate a predicted uplift measure. This uplift measure can be compared against the ground-truth uplift (determined at block 420), and the difference may be used to generate a loss term that is used to refine the model parameters via backpropagation. In some cases, the user's prior device (e.g., used prior to the switch) and/or subsequent device (e.g., used after the switch) can also be provided as input, alongside the user data.

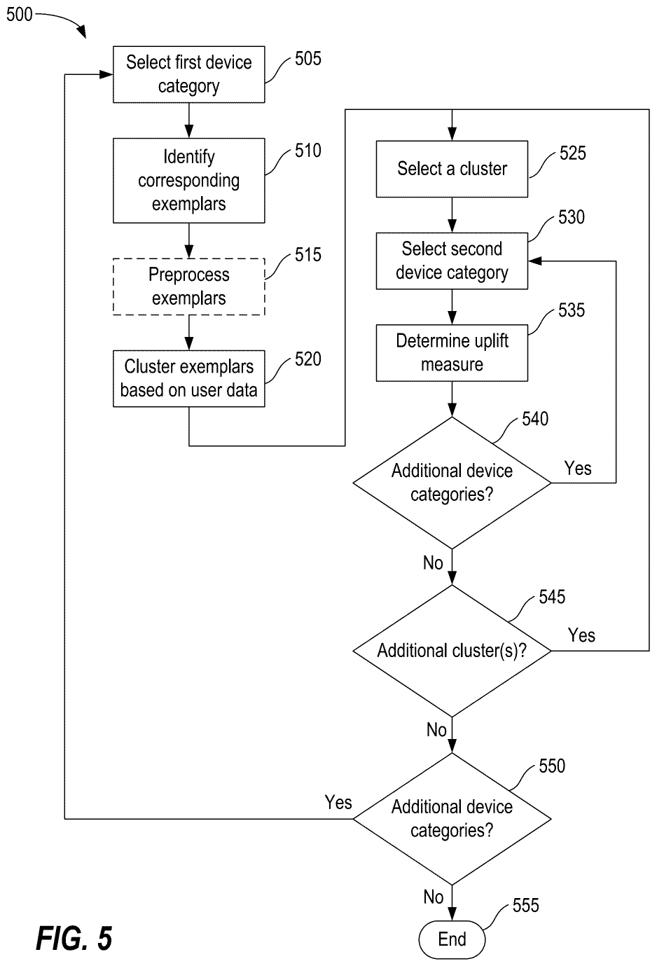

In some embodiments, the recommender system uses a cluster-based architecture for the machine learning model(s). For example, as discussed above with reference to FIG. 3, the recommender system may generate a plurality of clustering models (e.g., one for each “first device category” reflected in the training data), where each clustering model includes a set of clusters (generated based on user data from prior to the historical switches), and each cluster has a set of uplift measures (e.g., one or more for each “second device category” reflected in the training data). One example technique for training a clustering-based architecture is described in more detail below with reference to FIG. 5.

Generally, training the model(s) can continue until any number and variety of termination criteria are met, such as determining that all training exemplars have been used, determining that model accuracy has met a defined target (e.g., using test data), determining that a defined amount of time or resources have been spent training, and the like.

Additionally, in some embodiments, the machine learning model(s) may be retrained according to a variety of training criteria, including periodically (e.g., weekly), upon determining that the model performance or accuracy has degraded (e.g., determined using test data or feedback from current users), and the like.

At block 435, once the model(s) are trained, the recommender system deploys the machine learning models for runtime inferencing. As discussed above, deploying the model(s) generally includes performing one or more operations to prepare them for inferencing. For example, the recommender system may deploy the model(s) locally (to perform inferencing locally), transmit or otherwise provide the model(s) to one or more remote systems for inferencing, and the like.

### Example Method for Training a Set of Clustering Models to Generate Predicted Uplift Measures

FIG. 5 is a flow diagram depicting a method 500 for training a set of clustering models to generate predicted uplift measures. In some embodiments, the method 500 is performed by a recommender system, such as the recommender system 125 of FIG. 1. In at least one embodiment, the method 500 provides additional detail for block 430 of FIG. 4, and/or for the architecture 300 of FIG. 3.

At block 505, the recommender system selects a first device category or type, from a set of defined types or categories of devices that the recommender system is configured to suggest or recommend. As discussed above, in embodiments, the defined set of device categories can generally include any number of different discrete categories, and may be defined using any suitable characteristic or criteria for the devices. For example, the device categories may correspond to how the device is used or designed (e.g., full-face PAP masks, nasal-only PAP masks, pillow PAP masks, oral-only PAP masks, hybrid PAP masks, and the like), the specific model or name of the device, the genre or style, and the like.

Generally, at block 505, the recommender system may select the first device category using any suitable criteria, including randomly or pseudo-randomly, as each of the device categories will be processed using the method 500. Additionally, though a sequential process (processing each first device category in turn) is depicted for conceptual clarity, in some embodiments, the recommender system can process some or all of the device categories in parallel.

At block 510, the recommender system identifies a set of training exemplars that correspond to the selected first device category. For example, as discussed above, the recommender system may identify the training exemplars that specify the selected device category as the “first” or “initial” device category (e.g., the device category that the user was using prior to the switch associated with the exemplar). By generating a discrete clustering model for each such “first” category, the recommender system can enable efficient inferencing (e.g., where the appropriate model is selected based on the category of the device that is currently used by the user requesting a new device).

At block 515, the recommender system can optionally perform one or more preprocessing operations on and/or using the selected set of exemplars. For example, in some embodiments, the recommender system can apply one or more dimensionality reduction techniques to the exemplars to reduce the number of features (e.g., to reduce the exemplars from four features to two features). Such reduction may facilitate visualization of the resulting clusters (e.g., allowing them to be depicted on a two-dimensional graph or image), as well as reducing computational expense of performing the clustering (e.g., because clustering on fewer dimensions is generally less computationally expense than clustering on more dimensions).

In some embodiments, the preprocessing can include performing one or more operations to determine the optimal number of clusters. For example, the recommender system may use a user-defined number of clusters, or may perform one or more operations to infer or identify the optimal number. In at least one embodiment, the recommender system can cluster the selected exemplars multiple times, using a different number of clusters each time (e.g., using anywhere from five to twenty clusters). The recommender system can then evaluate the quality of each set of clusters (e.g., based on the silhouette scores to determine how well-separated the clusters are) to identify what number of clusters is optimal for the specific set of exemplars identified at block 510.

At block 520, the recommender system then clusters the exemplars based on the underlying user data for each. For example, as discussed above, the recommender system may use features such as the user's demographics and/or the usage data (e.g., average usage duration, AHI, mask leak, and the like) from a defined window of time prior to the switch as input features in order to cluster the users/device switches. In this way, exemplars within each cluster may generally be considered to be more similar to the other exemplars within the cluster, as compared to exemplars in other clusters. That is, when a new user is assigned to one or more clusters, the exemplars or data points in the cluster(s) may be identified as similar (and the corresponding uplift measures may be used as indicative or predictive).

As discussed above, the recommender system can generally use any suitable clustering approach, including probabilistic clustering techniques (e.g., Gaussian mixture), binary or non-probabilistic techniques (e.g., k-means), and so on. In an embodiment, clustering the exemplars at block 520 may generally be referred to as generating a machine learning model and/or clustering model, training or refining a machine learning model or clustering model, and the like (e.g., block 520 may correspond to creating a clustering model 305 of FIG. 3, with a corresponding set of clusters 310). In some embodiments, generating, training, and/or refining the machine learning models/clustering models may further include additional operations, such as those discussed below with reference to blocks 525, 530, 535, 540, 545, and 550.

At block 525, the recommender system selects one of the clusters in the clustering model generated at block 520. Generally, at block 525, the recommender system may select the cluster using any suitable criteria, including randomly or pseudo-randomly, as each of the clusters will be processed using the method 500. Additionally, though a sequential process (processing each cluster in turn) is depicted for conceptual clarity, in some embodiments, the recommender system can process some or all of the clusters in parallel.

At block 530, the recommender system selects a second device category, from the set of defined types or categories of devices that the recommender system is configured to suggest or recommend. Generally, at block 530, the recommender system may select the second device category using any suitable criteria, including randomly or pseudo-randomly, as each of the device categories will be processed using the method 500. Additionally, though a sequential process (processing each second device category in turn) is depicted for conceptual clarity, in some embodiments, the recommender system can process some or all of the device categories in parallel.

In an embodiment, as discussed above, the second device category may also be referred to as a new device category, an alternative device category, and the like. In at least one embodiment, the second device category is selected from a set of categories that are reflected in the identified set of training exemplars. That is, the set of second device categories may include any category that a user (who was originally using the first category selected at block 505) switched to, as reflected in the historical data. In such an embodiment, the recommender system may thereby refrain from selecting or processing any device categories that were not reflected in the training data (e.g., if no users switched from a pillow mask to a nasal-only mask), and may similarly refrain from selecting the same device category that was selected at block 505 (e.g., because “switching” from the first device category to the first device category is not, in fact, a switch).

At block 535, the recommender system determines an uplift measure for the selected second device category with respect to the exemplars included in the selected cluster. For example, the recommender system may identify all exemplars or data points, in the selected cluster, that indicate or are associated with the selected second device category. The recommender system can then aggregate the respective uplift measure(s) from each (e.g., by determining the average or median value of them) in order to generate an aggregate or predictive uplift measure with respect to the cluster and device type. That is, the aggregated uplift measure(s) can reflect the aggregate (e.g., median) uplift experienced by a similar set of users (defined by the selected cluster) when switching from the first device category (selected at block 505) to the second device category (selected at block 530).

At block 540, the recommender system determines whether there is at least one additional second device category that has not-yet been processed with respect to the selected cluster (selected at block 525). If so the method 500 returns to block 530. In this way, the recommender system generates the aggregated uplift measures (e.g., uplift measures 315 of FIG. 3) for each second device category, with respect to the first device category and cluster. If no additional second device categories remain, the method 500 continues to block 545. At block 545, the recommender system determines whether there is at least one additional cluster in the clustering model (generated at block 520) that has not-yet been selected for processing. If so, the method 500 returns to block 525. If not, the method 500 continues to block 550.

At block 550, the recommender system determines whether there is at least one additional first device category (from the initial set of device categories considered at block 505) that has not-yet been processed to generate a clustering model. If so, the method 500 returns to block 505 to select a new first device category. If not, the method 500 terminates at block 555, and the model(s) can be ensembled and/or deployed for runtime use.

### Example Method for Using Machine Learning to Drive Device Switches

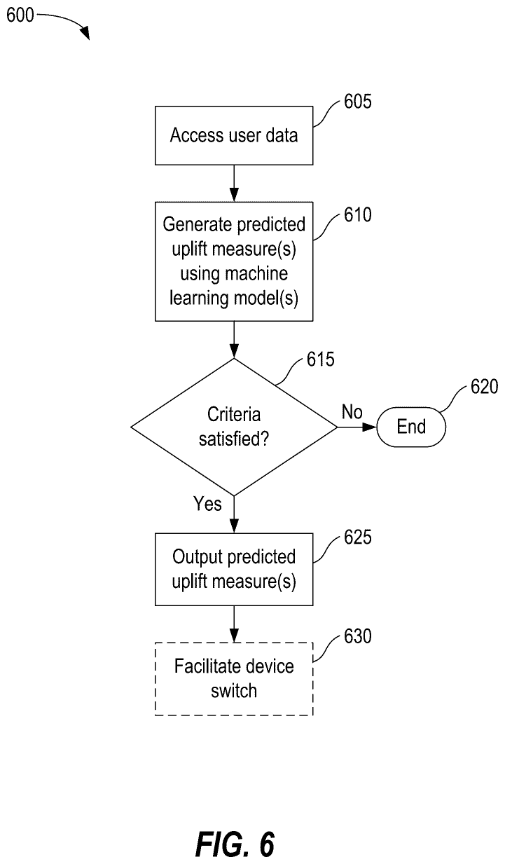

FIG. 6 is a flow diagram depicting a method 600 for using machine learning to drive device switches. In some embodiments, the method 600 is performed by a recommender system, such as the recommender system 125 of FIG. 1. In at least one embodiment, the method 600 is used to perform inferencing using machine learning models, such as those trained using the method 400 of FIG. 4 and/or the method 500 of FIG. 5.

At block 605, the recommender system accesses user data (e.g., user data 115 and/or device 110 of FIG. 1) for a current user. For example, as discussed above, the recommender system may access the user data based on determining that the user may need or benefit from a new device category (e.g., because their usage statistics are below a defined threshold or meet other defined criteria, because the user or a third party, such as a doctor, has indicated that the user may benefit from a new device, and the like). In some embodiments, the recommender system accesses the user data based on other criteria, such as if the recommender system is configured to periodically evaluate the user data to identify users that may benefit from a device switch.

As discussed above, the user data generally includes usage information and/or user characteristics/demographics for the user. For example, the user data may include the user's age, gender, weight, and the like. Similarly, the user data may include the user's usage data, with respect to their current device/device category, for some defined window of time (e.g., the last seven days). For example, in a PAP therapy embodiment, the user data may include information such as the average usage duration, average AHI, and the like.

At block 610, the recommender system generates one or more predicted uplift measures for the user by processing the user data using one or more machine learning models. Generally, the particular techniques used to generate the predicted uplift measure(s) may vary depending on the particular implementation and model architecture used. For example, if a regression model (e.g., a neural network) is used, the recommender system may process the user data and an indication of the current user device (used by the user) to generate one or more uplift measures (e.g., a set of output predictions, each corresponding to a given second device category).

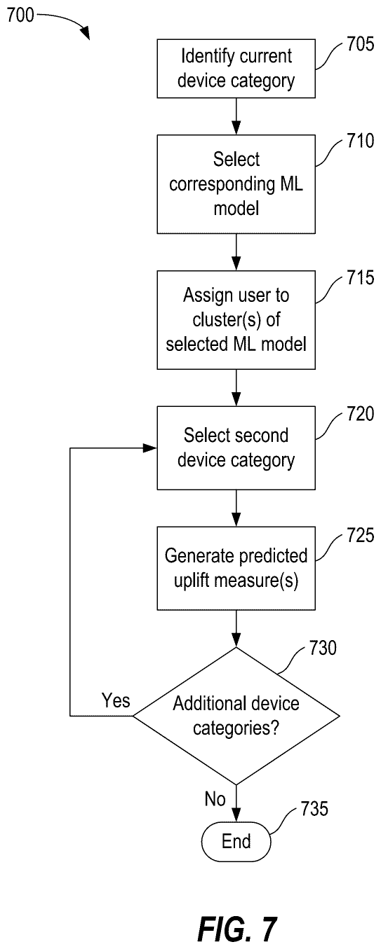

In one embodiment, the recommender system uses a set of regression models, where each is trained on/corresponds to a respective device type. For example, each model may be trained with respect to a given second device, such that, when the first device category is provided as input, the output measure indicates the predicted uplift if the user switches from the first device to the given second category. As another example, each model may be trained with respect to a given first device, such that, when a proposed second device category is provided as input, the output measure indicates the predicted uplift if the user switches from the given first device to the proposed second category. In at least one embodiment, the recommender system may train a single model to receive both the first and second device categories as input in order to generate a single uplift measure. In such an embodiment, the recommender system can iteratively process each possible second device category using the model in order to generate the set of uplift measures. Further, in some embodiments, the recommender system uses a clustering-based approach (e.g., the architecture 300 of FIG. 3). One example of generating predicted uplift measures using such a clustering architecture is discussed below with reference to FIG. 7.

At block 615, the recommender system determines whether one or more defined uplift criteria are satisfied. In some embodiments, the criteria specify a minimum or threshold uplift measure, such that the recommender system can proceed with further steps if one or more uplift measures are sufficiently positive, and refrain from further operations if all of the uplift measures do not meet the threshold (e.g., because switching device categories for the user is unlikely to return substantial benefit). In this way, the recommender system can selectively proceed with the method 600 based on the generated measures, thereby reducing computational expense and confusion. In some embodiments, the recommender system may nevertheless proceed to output the predicted uplift measures regardless of whether it meets the threshold (e.g., the system may not use any criteria or thresholds at all).

In at least one embodiment, the applicable criteria may differ depending on the triggering condition that initiated performance of the method 600. For example, if the recommender system began the method 600 automatically (e.g., evaluating each user periodically), the recommender system may use a relatively high uplift threshold to determine whether to take further steps or to refrain from further processing, as compared to the threshold used when a user or third party manually initiates the method 600.

If, at block 615, the recommender system determines that the criteria are not satisfied, the method 600 terminates at block 620. In at least one embodiment, the recommender system can output or provide an indication that no switch is recommended (while optionally including one or more uplift measure(s)). If the recommender system determines that the criteria are satisfied, the method 600 continues to block 625, where the recommender system outputs the predicted uplift measure(s).

For example, the recommender system may output some or all of the predicted uplift measures for the user via a graphical user interface (GUI). Generally, outputting the uplift measures at block 625 may include outputting them directly (e.g., via a GUI of the recommender system), and/or transmitting or otherwise providing them to another system or device. The other system or device can then output the measure(s) via a GUI on the respective device.

Generally, outputting the measures may include displaying them on a GUI associated with or used by user themselves (e.g., on a smartphone, application, computer, or other device used by the user), and/or on a GUI of a third party (e.g., a healthcare provider or professional), such as the computer or other device used by a doctor to generate the recommendations.

In some embodiments, prior to outputting the uplift measures, the recommender system can perform one or more preprocessing operations, such as to rank or sort them (e.g., with higher uplift measures near the top of the list or in a more prominent portion of the GUI), filtering them (e.g., to remove any uplift measures below a threshold), and the like. In embodiments, the recommender system may output the highest measure, the highest N measures, any measures above a threshold, and the like.

In some embodiments, in addition to or instead of outputting the predicted uplift measure(s), the recommender system can output an indication of the recommended alternative or second device category or categories (e.g., the highest-scored, the N highest-scored, or those with a score above a threshold). In at least one embodiment, the specific data output may vary depending on the receiving entity. For example, if the recommender system outputs data directly to the user, the recommender system may output the highest-scored alternative device category (or a reduced list of the highest-scored alternatives). If the recommender system outputs the data to a healthcare provider, the recommender system may further include information such as the predicted uplift measure(s) themselves.

At block 630, the recommender system can optionally facilitate a device switch based on the predicted uplift measure(s). Generally, facilitating the device switch can include a wide variety of operations to implement the recommendation, such as creating an order, instruction, or prescription to ship or otherwise provide a device of the recommended category to the user, dispensing the recommended category (e.g., via an automated dispensing system), and the like. In some embodiments, the recommender system facilitates the device switch in response to receiving approval or selection of a recommended second device (e.g., from the user and/or a third party). In at least one embodiment, the recommender system facilitates the switch automatically, such as in response to determining that the predicted output measure exceeds some defined threshold.

### Example Method for Using a Set of Clustering Models to Generate Predicted Uplift Scores

FIG. 7 is a flow diagram depicting a method 700 for using a set of clustering models to generate predicted uplift scores. In some embodiments, the method 700 is performed by a recommender system, such as the recommender system 125 of FIG. 1. In at least one embodiment, the method 700 provides additional detail for block 610 of FIG. 6, and/or for the architecture 300 of FIG. 3.

At block 705, the recommender system identifies the current or first device category. That is, the recommender system identifies or determines the device that the user is currently using or associated with. For example, in the PAP example, the recommender system may identify which device (or device category) the user was most-recently provided, such as a full-face mask, a nasal mask, and the like.

At block 710, the recommender system identifies and selects the corresponding ML model associated with the current device category (e.g., the clustering model, generated at block 520, trained for the first device category). For example, the recommender system may select a clustering model 305 of FIG. 3. By using category-specific models, the recommender system can significantly improve model accuracy and explainability while reducing computational expense of inferencing.

At block 715, the recommender system assigns the user to one or more clusters of the selected model, based on the user's data. That is, based on the user data (such as their average usage duration, average AHI, and the like) for a window of time (e.g., the last seven days) and/or other user characteristics (such as their age), the recommender system can identify which cluster(s) in the selected clustering model the user should be assigned to. In some embodiments, if the clusters were generated based on reduced dimensionality data, the recommender system can similarly apply the dimensionality reduction operation(s) to the user data prior to assigning them to cluster(s).

In an embodiment, whether the user is assigned to a single cluster or multiple clusters may depend on the particular implementation and architecture. For example, if k-means or other similar techniques were used to generate the clusters, the recommender system may assign the user to a single cluster (e.g., the cluster having a center that is nearest to the user's data in the multidimensional cluster space). In some embodiments, if probabilistic techniques such as Gaussian mixture models were used to perform the clustering, the recommender system may effectively assign the user to all of the clusters by generating, for each respective cluster, a probability that the user should be assigned to the respective cluster (e.g., based on the distance between the user's data and the respective cluster center).

At block 720, the recommender system selects a second device category (e.g., an alternative or other device category, different from the first or current device category determined at block 705) to which the user may switch. For example, if the device categories include nasal PAP masks, full-face PAP masks, and pillow PAP masks, and the user currently uses a nasal mask, the recommender system may select either the full-face mask category or the pillow mask category. Generally, at block 720, the recommender system may select the second device category using any suitable criteria, including randomly or pseudo-randomly, as each of the device categories will be processed using the method 700. Additionally, though a sequential process (processing each second device category in turn) is depicted for conceptual clarity, in some embodiments, the recommender system can process some or all of the device categories in parallel.

At block 725, the recommender system generates a predicted uplift measure, with respect to the second device category, based on the measure(s) indicated for the cluster(s) to which the user was assigned. For example, the predicted uplift measure may be generated based on the aggregated uplift measures determined at block 535 of FIG. 5. In one embodiment, if the user was assigned to a single cluster, the recommender system may identify or generate the aggregate measure for the cluster and category, and use this aggregated uplift measure (with respect to the cluster and device category) as the predicted uplift measure for the current user.

In some embodiments, if the user is assigned to multiple clusters, the recommender system may compute a weighted average of the uplift measures indicated in each cluster (e.g., of the median uplift indicated for each cluster), where the weight is defined based on the probability that the user belongs to each respective cluster. For example, suppose the assigned probability for a first cluster is 0.3, the probability for a second cluster is 0.7, the first cluster has a predicted uplift measure of 2, and the second cluster has a predicted uplift measure of 1. In an embodiment, the recommender system may generate the predicted uplift measure for the user as (0.3*2)+(0.7*1)=1.3.

At block 730, the recommender system determines whether there is at least one additional second device category remaining for which a predicted uplift measure has not been generated. If so, the method 700 returns to block 720. If not, the method 700 terminates at block 735, and the predicted uplift measure(s) can be output or otherwise used, as discussed above.

### Example Method for Facilitating the Provisioning of Devices Based on Predicted Uplift Measures

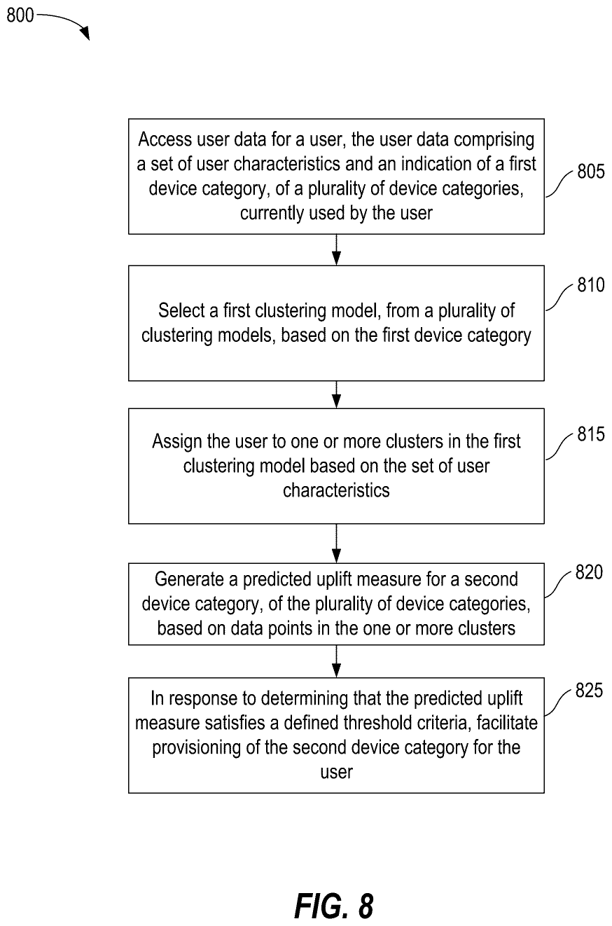

FIG. 8 is a flow diagram depicting a method 800 for facilitating the provisioning of devices based on predicted uplift measures. In some embodiments, the method 800 is performed by a recommender system, such as the recommender system 125 of FIG. 1.

At block 805, user data (e.g., user data 115 of FIG. 1) for a user is accessed, the user data comprising a set of user characteristics and an indication of a first device category (e.g., device 110 of FIG. 1), of a plurality of device categories, currently used by the user.

At block 810, a first clustering model is selected, from a plurality of clustering models (e.g., machine learning models 140 of FIG. 1, and/or clustering models 305 of FIG. 3), based on the first device category.

At block 815, the user is assigned to one or more clusters (e.g., clusters 310 of FIG. 3) in the first clustering model based on the set of user characteristics.

At block 820, a predicted uplift measure (e.g., uplift measures 315 of FIG. 3) for a second device category, of the plurality of device categories, is generated based on data points in the one or more clusters.

At block 825, in response to determining that the predicted uplift measure satisfies one or more defined criteria, provisioning of the second device category (e.g., device 145 of FIG. 1) for the user is facilitated.

### Example Method for Generating Predicted Uplift Measures for PAP Devices and Medical Providers

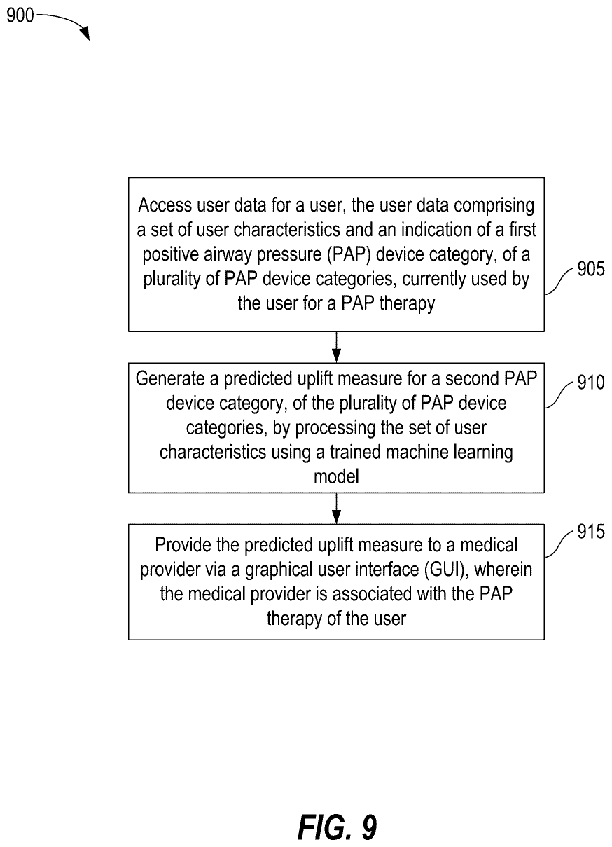

FIG. 9 is a flow diagram depicting a method 900 for generating predicted uplift measures for PAP devices and medical providers. In some embodiments, the method 900 is performed by a recommender system, such as the recommender system 125 of FIG. 1.

At block 905, user data (e.g., user data 115 of FIG. 1) for a user is accessed, the user data comprising a set of user characteristics and an indication of a first positive airway pressure (PAP) device category (e.g., device 110 of FIG. 1), of a plurality of PAP device categories, currently used by the user for a PAP therapy.

At block 910, a predicted uplift measure for a second PAP device category (e.g., device 145 of FIG. 1), of the plurality of PAP device categories, is generated by processing the set of user characteristics using a trained machine learning model (e.g., machine learning model(s) 140 of FIG. 1).

At block 915, the predicted uplift measure is provided to a medical provider via a graphical user interface (GUI), wherein the medical provider is associated with the PAP therapy of the user.

### Example Method for Generating Predicted Uplift Measures for Pap Devices and Users

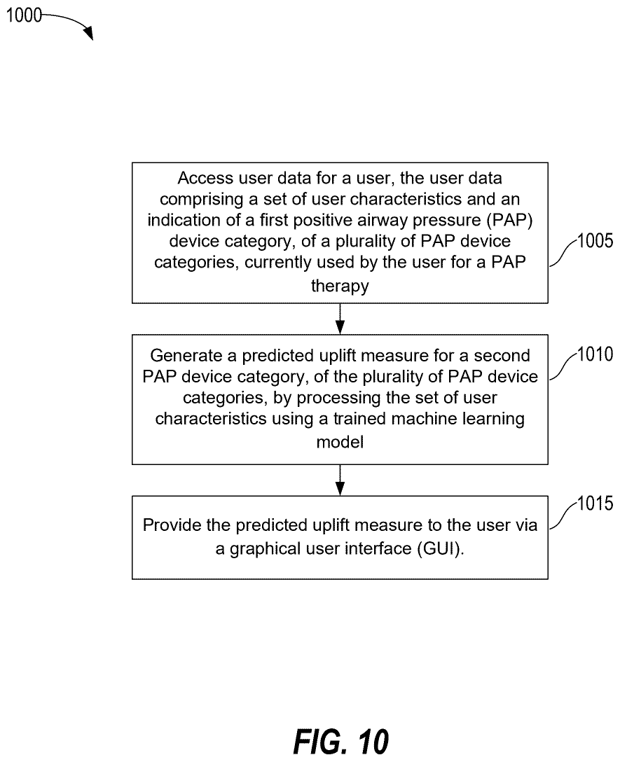

FIG. 10 is a flow diagram depicting a method 1000 for generating predicted uplift measures for PAP devices and users. In some embodiments, the method 1000 is performed by a recommender system, such as the recommender system 125 of FIG. 1.

At block 1005, user data (e.g., user data 115 of FIG. 1) for a user is accessed, the user data comprising a set of user characteristics and an indication of a first positive airway pressure (PAP) device category (e.g., device 110 of FIG. 1), of a plurality of PAP device categories, currently used by the user for a PAP therapy.

At block 1010, a predicted uplift measure for a second PAP device category (e.g., device 145 of FIG. 1), of the plurality of PAP device categories, is generated by processing the set of user characteristics using a trained machine learning model (e.g., machine learning model(s) 140 of FIG. 1).

At block 1015, the predicted uplift measure is provided to the user via a graphical user interface (GUI).

### Example Computing Device for Object Recommendation

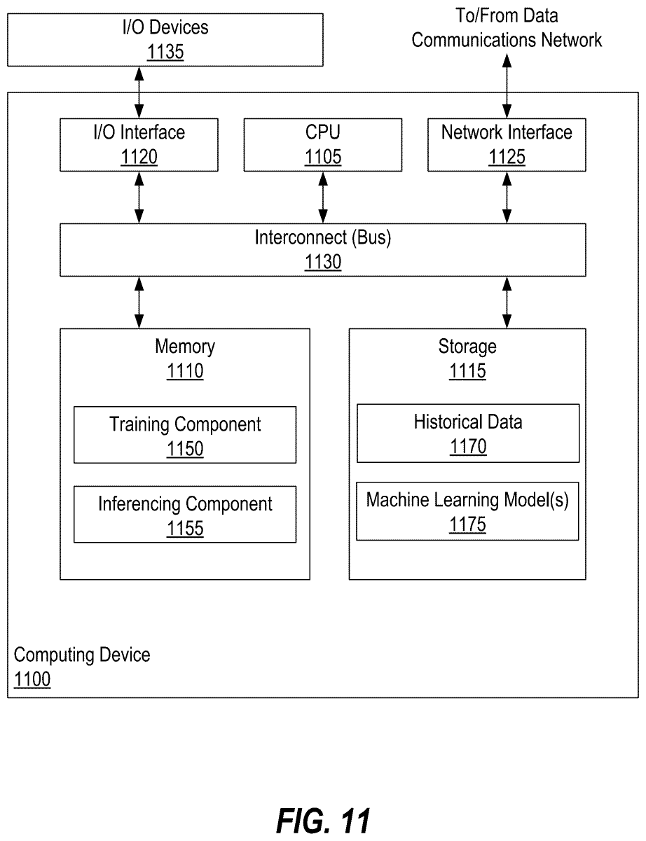

FIG. 11 depicts an example computing device 1100 configured to perform various embodiments of the present disclosure. Although depicted as a physical device, in embodiments, the computing device 1100 may be implemented using virtual device(s), and/or across a number of devices (e.g., in a cloud environment). In one embodiment, the computing device 1100 corresponds to a recommender system (e.g., recommender system 125 of FIG. 1).

As illustrated, the computing device 1100 includes a CPU 1105, memory 1110, storage 1115, a network interface 1125, and one or more I/O interfaces 1120. In the illustrated embodiment, the CPU 1105 retrieves and executes programming instructions stored in memory 1110, as well as stores and retrieves application data residing in storage 1115. The CPU 1105 is generally representative of a single CPU and/or GPU, multiple CPUs and/or GPUs, a single CPU and/or GPU having multiple processing cores, and the like. The memory 1110 is generally included to be representative of a random access memory. Storage 1115 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, I/O devices 1135 (such as keyboards, monitors, etc.) are connected via the I/O interface(s) 1120. Further, via the network interface 1125, the computing device 1100 can be communicatively coupled with one or more other devices and components (e.g., via a network, which may include the Internet, local network(s), and the like). As illustrated, the CPU 1105, memory 1110, storage 1115, network interface(s) 1125, and I/O interface(s) 1120 are communicatively coupled by one or more buses 1130.

In the illustrated embodiment, the memory 1110 includes a training component 1150 and an inferencing component 1155, which may perform one or more embodiments discussed above. Although depicted as discrete components for conceptual clarity, in embodiments, the operations of the depicted components (and others not illustrated) may be combined or distributed across any number of components. Further, although depicted as software residing in memory 1110, in embodiments, the operations of the depicted components (and others not illustrated) may be implemented using hardware, software, or a combination of hardware and software.

In one embodiment, the training component 1150 may be used to train machine learning models (e.g., machine learning models 1175) to generate predicted uplift measures based on historical data (e.g., historical data 1170), as discussed above. For example, the training component 1150 (which may correspond to the training component 130 of FIG. 1) may evaluate the historical data 1170 to identify historical switch events, extract the corresponding data (e.g., from windows of time just before and after the switch), preprocess the data if needed, and use the data to train one or more models (e.g., regression models, clustering models, and the like). In some embodiments, the training component 1150 can use techniques such as the method 400 of FIG. 4 and/or the method 500 of FIG. 5 to train the machine learning model(s) 1175.

In one embodiment, the inferencing component 1155 may be used to generate predicted uplift measures using trained models and/or to output or facilitate switching to suggested or recommended alternative device categories, as discussed above. For example, the inferencing component 1155 (which may correspond to the inferencing component 135 of FIG. 1) may use the trained machine learning models 1175 to generate a predicted uplift score for each alternative device category based on user data and the user's current device category. In some embodiments, the inferencing component 1155 can use techniques such as the method 600 of FIG. 6, the method 700 of FIG. 7, the method 800 of FIG. 8, the method 900 of FIG. 9, and/or the method 1000 of FIG. 10, to generate the predicted uplift measure(s).

In the illustrated example, the storage 1115 includes historical data 1170 and one or more machine learning models 1175. In one embodiment, the historical data 1170 (which may correspond to the historical data 120 of FIG. 1) generally includes information relating to historical device switches, as discussed above. The machine learning models 1175 (which may correspond to the machine learning models 140 of FIG. 1 and/or the architecture 300 of FIG. 3) can generally be used to generate predicted uplift measures based on user data.

Although depicted as residing in storage 1115 for conceptual clarity, the historical data 1170 and machine learning models 1175 may be stored in any suitable location, including memory 1110 or in one or more remote systems distinct from the computing device 1100.

### Additional Considerations

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the embodiments set forth herein. In addition, the scope of the disclosure is intended to cover such an apparatus or method that is practiced using other structure, functionality, or structure and functionality in addition to, or other than, the various embodiments of the disclosure set forth herein. It should be understood that any embodiment of the disclosure disclosed herein may be embodied by one or more elements of a claim.

As used herein, the word “exemplary” means “serving as an example, instance, or illustration.” Any embodiment described herein as “exemplary” is not necessarily to be construed as preferred or advantageous over other embodiments.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

Embodiments of the invention may be provided to end users through a cloud computing infrastructure. Cloud computing generally refers to the provision of scalable computing resources as a service over a network. More formally, cloud computing may be defined as a computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction. Thus, cloud computing allows a user to access virtual computing resources (e.g., storage, data, applications, and even complete virtualized computing systems) in “the cloud,” without regard for the underlying physical systems (or locations of those systems) used to provide the computing resources.

Typically, cloud computing resources are provided to a user on a pay-per-use basis, where users are charged only for the computing resources actually used (e.g. an amount of storage space consumed by a user or a number of virtualized systems instantiated by the user). A user can access any of the resources that reside in the cloud at any time, and from anywhere across the Internet. In context of the present invention, a user may access applications or systems (e.g., recommender system 125 of FIG. 1) or related data available in the cloud. For example, the recommender system could execute on a computing system in the cloud and train/use machine learning models to drive device suggestions. In such a case, the recommender system could maintain the models in the cloud, and use them to drive improved device recommendations. Doing so allows a user to access this information from any computing system attached to a network connected to the cloud (e.g., the Internet).

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various embodiments described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

### EXAMPLE CLAUSES

Implementation examples are described in the following numbered clauses:
Clause 1: A method, comprising: accessing user data for a user, the user data comprising a set of user characteristics and an indication of a first device category, of a plurality of device categories, currently used by the user; selecting a first clustering model, from a plurality of clustering models, based on the first device category; assigning the user to one or more clusters in the first clustering model based on the set of user characteristics; generating a predicted uplift measure for a second device category, of the plurality of device categories, based on data points in the one or more clusters; and in response to determining that the predicted uplift measure satisfies one or more defined criteria, facilitating provisioning of the second device category for the user.
Clause 2: The method of Clause 1, wherein generating the predicted uplift measure comprises: identifying a set of data points in the one or more clusters, each data point in the set of data points corresponding to a prior user switching to the second device category; and determining an aggregate uplift measure based on the identified set of data points.
Clause 3: The method of any one of Clauses 1-2, wherein assigning the user to the one or more clusters comprises, for each respective cluster of a plurality of clusters in the first clustering model, generating a respective probability that the user belongs to the respective cluster.
Clause 4: The method of any one of Clauses 1-3, wherein generating the predicted uplift measure comprises: determining, for each respective cluster of the plurality of clusters, a respective aggregate uplift measure for the second device category; and computing a weighted average of the aggregate uplift measures based on the generated probabilities.
Clause 5: The method of any one of Clauses 1-4, wherein each respective clustering model in the plurality of clustering models corresponds to a respective device category of the plurality of device categories.
Clause 6: The method of any one of Clauses 1-5, wherein: the plurality of device categories correspond to positive airway pressure (PAP) device categories, and the PAP device categories are defined based at least in part on device types.
Clause 7: The method of any one of Clauses 1-6, wherein the device types comprise: a full face device type, a nasal device type, and a pillow device type.
Clause 8: The method of any one of Clauses 1-7, wherein the set of user characteristics comprise at least one of: (i) an age of the user; (ii) usage duration data for the user, with respect to the first device category, during a prior window of time; or (iii) an apnea hypopnea index (AHI) of the user during the prior window of time.
Clause 9: The method of any one of Clauses 1-8, wherein the predicted uplift measure indicates a predicted usage of the second device category, by the user, as compared to the first device category.
Clause 10: The method of any one of Clauses 1-9, wherein the first clustering model is generated by: identifying a set of prior users associated with the first device category; clustering the set of prior users based on corresponding prior user characteristics; and identifying a subset of prior users, from the set of prior users, that switched to the second device category; and determining an aggregate uplift measure based on the subset of prior users.
Clause 11: The method of any one of Clauses 1-10, wherein determining the aggregate uplift measure comprises, for at least a first prior user of the subset of prior users: estimating a switch date corresponding to when the first prior user switched from the first device category to the second device category; determining first usage duration data for the first prior user during a first window of time prior to the switch date; and determining second usage duration data for the first prior user during a second window of time subsequent to the switch date, wherein the first and second windows of time are non-adjacent and do not include the switch date.
Clause 12: A method, comprising: accessing user data for a user, the user data comprising a set of user characteristics and an indication of a first positive airway pressure (PAP) device category, of a plurality of PAP device categories, currently used by the user for a PAP therapy; generating a predicted uplift measure for a second PAP device category, of the plurality of PAP device categories, by processing the set of user characteristics using a trained machine learning model; and providing the predicted uplift measure to a medical provider via a graphical user interface (GUI), wherein the medical provider is associated with the PAP therapy of the user.
Clause 13: The method of Clause 12, wherein generating the predicted uplift measure comprises: selecting a first clustering model, from a plurality of clustering models, based on the first PAP device category; assigning the user to one or more clusters in the first clustering model based on the set of user characteristics; generating a predicted uplift measure for the second PAP device category, of the plurality of PAP device categories, based on data points in the one or more clusters; and in response to determining that the predicted uplift measure satisfies one or more defined criteria, facilitating provisioning of the second PAP device category for the user.
Clause 14: The method of Clause 13, wherein generating the predicted uplift measure comprises: identifying a set of data points in the one or more clusters, each data point in the set of data points corresponding to a prior user switching to the second PAP device category; and determining an aggregate uplift measure based on the identified set of data points.
Clause 15: The method of any one of Clauses 13-14, wherein: assigning the user to the one or more clusters comprises, for each respective cluster of a plurality of clusters in the first clustering model, generating a respective probability that the user belongs to the respective cluster, and generating the predicted uplift measure comprises: determining, for each respective cluster of the plurality of clusters, a respective aggregate uplift measure for the second PAP device category; and computing a weighted average of the aggregate uplift measures based on the generated probabilities.
Clause 16: The method of any one of Clauses 13-15, wherein each respective clustering model in the plurality of clustering models corresponds to a respective PAP device category of the plurality of PAP device categories.
Clause 17: A method, comprising: accessing user data for a user, the user data comprising a set of user characteristics and an indication of a first positive airway pressure (PAP) device category, of a plurality of PAP device categories, currently used by the user for a PAP therapy; generating a predicted uplift measure for a second PAP device category, of the plurality of PAP device categories, by processing the set of user characteristics using a trained machine learning model; and providing the predicted uplift measure to the user via a graphical user interface (GUI).
Clause 18: The method of Clause 17, wherein generating the predicted uplift measure comprises: selecting a first clustering model, from a plurality of clustering models, based on the first PAP device category; assigning the user to one or more clusters in the first clustering model based on the set of user characteristics; generating a predicted uplift measure for the second PAP device category, of the plurality of PAP device categories, based on data points in the one or more clusters; and in response to determining that the predicted uplift measure satisfies one or more defined criteria, facilitating provisioning of the second PAP device category for the user.
Clause 19: The method of Clause 18, wherein generating the predicted uplift measure comprises: identifying a set of data points in the one or more clusters, each data point in the set of data points corresponding to a prior user switching to the second PAP device category; and determining an aggregate uplift measure based on the identified set of data points.
Clause 20: The method of any one of Clauses 18-19, wherein: assigning the user to the one or more clusters comprises, for each respective cluster of a plurality of clusters in the first clustering model, generating a respective probability that the user belongs to the respective cluster, and generating the predicted uplift measure comprises: determining, for each respective cluster of the plurality of clusters, a respective aggregate uplift measure for the second PAP device category; and computing a weighted average of the aggregate uplift measures based on the generated probabilities.
Clause 21: A system, comprising: a memory comprising computer-executable instructions; and one or more processors configured to execute the computer-executable instructions and cause the processing system to perform a method in accordance with any one of Clauses 1-20.

Clause 22: A system, comprising means for performing a method in accordance with any one of Clauses 1-20.

Clause 23: A non-transitory computer-readable medium comprising computer-executable instructions that, when executed by one or more processors of a processing system, cause the processing system to perform a method in accordance with any one of Clauses 1-20.

Clause 24: A computer program product embodied on a computer-readable storage medium comprising code for performing a method in accordance with any one of Clauses 1-20.

What is claimed is:

1. A method, comprising:
accessing user data for a user, the user data comprising a set of user characteristics and an indication of a first device category, of a plurality of device categories, currently used by the user;
selecting a first clustering model, from a plurality of clustering models, based on the first device category;
assigning the user to one or more clusters in the first clustering model based on the set of user characteristics;
generating a predicted uplift measure for a second device category, of the plurality of device categories, based on data points in the one or more clusters; and
in response to determining that the predicted uplift measure satisfies one or more defined criteria, facilitating provisioning of the second device category for the user.

2. The method of claim 1, wherein generating the predicted uplift measure comprises:
identifying a set of data points in the one or more clusters, each data point in the set of data points corresponding to a prior user switching to the second device category; and
determining an aggregate uplift measure based on the identified set of data points.

3. The method of claim 1, wherein assigning the user to the one or more clusters comprises, for each respective cluster of a plurality of clusters in the first clustering model, generating a respective probability that the user belongs to the respective cluster.

4. The method of claim 3, wherein generating the predicted uplift measure comprises:
determining, for each respective cluster of the plurality of clusters, a respective aggregate uplift measure for the second device category; and
computing a weighted average of the aggregate uplift measures based on the generated probabilities.

5. The method of claim 1, wherein each respective clustering model in the plurality of clustering models corresponds to a respective device category of the plurality of device categories.

6. The method of claim 1, wherein:
the plurality of device categories correspond to positive airway pressure (PAP) device categories, and
the PAP device categories are defined based at least in part on device types.

7. The method of claim 6, wherein the device types comprise: a full face device type, a nasal device type, or a pillow device type.

8. The method of claim 6, wherein the set of user characteristics comprise at least one of:
(i) an age of the user;
(ii) usage duration data for the user, with respect to the first device category, during a prior window of time; or
(iii) an apnea hypopnea index (AHI) of the user during the prior window of time.

9. The method of claim 1, wherein the predicted uplift measure indicates a predicted usage of the second device category, by the user, as compared to the first device category.

10. The method of claim 1, wherein the first clustering model is generated by:
identifying a set of prior users associated with the first device category;
clustering the set of prior users based on corresponding prior user characteristics; and
identifying a subset of prior users, from the set of prior users, that switched to the second device category; and
determining an aggregate uplift measure based on the subset of prior users.

11. The method of claim 10, wherein determining the aggregate uplift measure comprises, for at least a first prior user of the subset of prior users:
estimating a switch date corresponding to when the first prior user switched from the first device category to the second device category;
determining first usage duration data for the first prior user during a first window of time prior to the switch date; and
determining second usage duration data for the first prior user during a second window of time subsequent to the switch date, wherein the first and second windows of time are non-adjacent and do not include the switch date.

12. A method, comprising:
accessing user data for a user, the user data comprising a set of user characteristics and an indication of a first positive airway pressure (PAP) device category, of a plurality of PAP device categories, currently used by the first user for a PAP therapy;
generating a predicted uplift measure for a second PAP device category, of the plurality of PAP device categories, by processing the set of user characteristics using a trained machine learning model; and
providing the predicted uplift measure to a medical provider via a graphical user interface (GUI), wherein the medical provider is associated with the PAP therapy of the user.

13. The method of claim 12, wherein generating the predicted uplift measure comprises:
selecting a first clustering model, from a plurality of clustering models, based on the first PAP device category;
assigning the user to one or more clusters in the first clustering model based on the set of user characteristics;
generating a predicted uplift measure for the second PAP device category, of the plurality of PAP device categories, based on data points in the one or more clusters; and
in response to determining that the predicted uplift measure satisfies one or more defined criteria, facilitating provisioning of the second PAP device category for the user.

14. The method of claim 13, wherein generating the predicted uplift measure comprises:
identifying a set of data points in the one or more clusters, each data point in the set of data points corresponding to a prior user switching to the second PAP device category; and
determining an aggregate uplift measure based on the identified set of data points.

15. The method of claim 13, wherein:
assigning the user to the one or more clusters comprises, for each respective cluster of a plurality of clusters in the first clustering model, generating a respective probability that the user belongs to the respective cluster, and
generating the predicted uplift measure comprises:
determining, for each respective cluster of the plurality of clusters, a respective aggregate uplift measure for the second PAP device category; and
computing a weighted average of the aggregate uplift measures based on the generated probabilities.

16. The method of claim 13, wherein each respective clustering model in the plurality of clustering models corresponds to a respective PAP device category of the plurality of PAP device categories.

17. A method, comprising:
accessing user data for a user, the user data comprising a set of user characteristics and an indication of a first positive airway pressure (PAP) device category, of a plurality of PAP device categories, currently used by the user for a PAP therapy;
generating a predicted uplift measure for a second PAP device category, of the plurality of PAP device categories, by processing the set of user characteristics using a trained machine learning model; and
providing the predicted uplift measure to the user via a graphical user interface (GUI).

18. The method of claim 17, wherein generating the predicted uplift measure comprises:
selecting a first clustering model, from a plurality of clustering models, based on the first PAP device category;
assigning the user to one or more clusters in the first clustering model based on the set of user characteristics;
generating a predicted uplift measure for the second PAP device category, of the plurality of PAP device categories, based on data points in the one or more clusters; and
in response to determining that the predicted uplift measure satisfies one or more defined criteria, facilitating provisioning of the second PAP device category for the user.

19. The method of claim 18, wherein generating the predicted uplift measure comprises:
identifying a set of data points in the one or more clusters, each data point in the set of data points corresponding to a prior user switching to the second PAP device category; and
determining an aggregate uplift measure based on the identified set of data points.

20. The method of claim 18, wherein:
assigning the user to the one or more clusters comprises, for each respective cluster of a plurality of clusters in the first clustering model, generating a respective probability that the user belongs to the respective cluster, and
generating the predicted uplift measure comprises:
determining, for each respective cluster of the plurality of clusters, a respective aggregate uplift measure for the second PAP device category; and
computing a weighted average of the aggregate uplift measures based on the generated probabilities.
